(19) United States
(12) Patent Application Publication
(10) Pub. No.: US 2021/0198229 A1
(43) Pub. Date: Jul. 1, 2021

(54) FRAGRANCE AND FLAVOR MATERIALS FROM 1-(2-HYDROXY-4-METHYLCYCLOHEXYL)-ETHANONE

(71) Applicant: TAKASAGO INTERNATIONAL CORPORATION, Tokyo (JP)

(72) Inventors: Thalia S. Dickerson, Wanaque, NJ (US); Louis J. Lombardo, Washingtonville, NY (US); Michael E. Lankin, High Bridge, NJ (US)

(73) Assignee: TAKASAGO INTERNATIONAL CORPORATION, Tokyo (JP)

(21) Appl. No.: 16/978,957

(22) PCT Filed: Mar. 8, 2019
(86) PCT No.: PCT/US19/21450

§ 371 Date: Sep. 8, 2020

Related U.S. Application Data
(60) Provisional application No. 62/640,490, filed on Mar. 8, 2018.

(51) Int. Cl. C07D 319/08; C07D 317/20; C07C 43/115; A23L 27/20; C11B 9/00

(52) U.S. Cl. CPC C07D 319/08; C07D 317/20; C11B 9/008; A23L 27/2052; C07C 43/115

(57) ABSTRACT

The present disclosure is directed to 1-(2-hydroxy-4-methycyclohexyl)-ethanone derivatives having unique and desired flavor and/or fragrance characteristics, as well as the synthesis and application of the derivatives. The compounds of the present disclosure can be employed alone or incorporated as fragrance or flavor ingredients to modify or enhance already existing fragrance compositions, solvents, media, and the like.

### CROSS-REFERENCE TO RELATED APPLICATIONS

This application claims priority to U.S. Provisional Patent Application Ser. No. 62/640,490, filed Mar. 8, 2018, the contents of which are incorporated by reference herein in its entirety.

### FIELD

The present application relates to compounds useful as fragrance or flavor components in fragrance or flavor compositions.

### BACKGROUND

There is a continuing interest in the preparation of new synthetic fragrance and flavor components with unique organoleptic properties and their use in consumer products. Differences in chemical structures can significantly impact odor, notes, and other organoleptic, chemical, and physical characteristics. Thus, there is a continuous need for new chemical structures that have favorable organoleptic properties. Once identified, these novel chemical compounds can provide perfumers and other persons with the ability to create new, unique fragrances.

There is also a need for more efficient synthesis techniques to prepare components with pleasing and consumer preferred fragrance and flavor compositions. One strategy to prepare such compounds is to make derivatives of a chemical entity. 1-(2-hydroxy-4-methylcyclohexyl)-ethanone is such a chemical entity.

The presently disclosed subject matter addresses these and other needs by providing new fragrance compounds with unique and desirable organoleptic properties, as discussed in detail below. Additionally, synthetic procedures for making such compounds are disclosed herein.

### SUMMARY

The present disclosure is directed to derivatives of 1-(2-hydroxy-4-methylcyclohexyl)-ethanone having unique and desired organoleptic characteristics, and synthesis and uses thereof. The compounds of the present disclosure can be employed alone or in combination with additional compounds. The compounds of the present disclosure can be incorporated into fragrance or flavor compositions.

In certain embodiments, the presently disclosed subject matter provides the compound represented by Formula I:

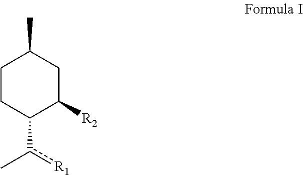

wherein R₁ is one of O, —OH, —NOH—NOR, or NOC(O)R;
wherein R₂ is one of —OC(O)R, —OH, or —OTHP; and
wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

In certain embodiments, the present disclosure provides compound of Formula I, wherein R₁ is one of —OH, —NOH—NOR, or NOC(O)R; and wherein R₂ is —OH.

In certain embodiments, the presently disclosed subject matter provides the compound represented by Formula Ia:

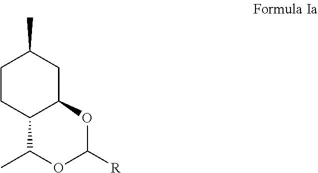

wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

In certain embodiments, the presently disclosed subject matter provides the compound represented by Formula Ib:

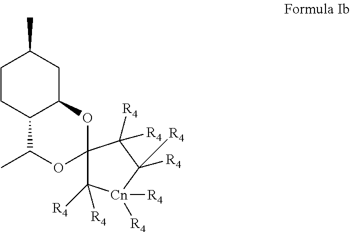

wherein n is 1, 2, 3, or 4;
wherein each R₄ is independently H or C₁-C₅ alkyl.

The present disclosure also provides a compound represented by Formula II:

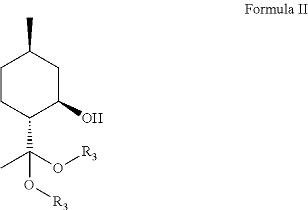

wherein each R₃ is independently H, C₁-C₈ alkyl, C₁-C₈ branched alkyl, C₁-C₈ alkenyl or C₁-C₈ branched alkenyl.

In certain embodiments, the presently disclosed subject matter provides the compound represented by Formula IIa:

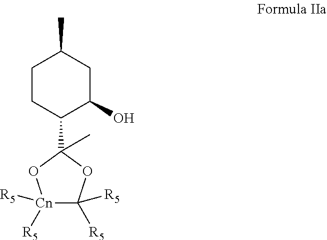

wherein n is 1 or 2;

wherein each R₅ is independently H, C₁-C₈ alkyl, C₁-C₈ branched alkyl, C₁-C₈ alkenyl or C₁-C₈ branched alkenyl.

The presently disclosed subject matter also provides for fragrance compositions comprising at least one compound disclosed herein. In a particular embodiment of a fragrance composition, the concentration of the at least one compound is from about 0.001% to about 20% by weight of the fragrance composition.

In certain embodiments, the fragrance composition further comprises one or more compounds selected from the group consisting of one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

The presently disclosed subject matter also provides for flavor compositions comprising at least one compound disclosed herein. In a particular embodiment of a flavor composition, the concentration of the at least one compound is from about 0.0001% to about 20% by weight of the flavor composition. In certain embodiments, the flavor composition further comprises a flavor carrier.

In certain embodiments, the compounds include constitutional isomers, enantiomers, stereoisomers, and racemic mixtures of said compounds listed herein.

Another aspect of the present disclosure provides a fragrance or flavor composition for addition to a consumer product comprising one or more of the presently disclosed compounds in an amount effective to impart a fragrance or flavor to the consumer product.

### DETAILED DESCRIPTION

As noted above, there remains a need and demand in the art for unique fragrance and flavor compositions. There is also a need for more efficient synthesis techniques to prepare components with pleasing and consumer preferred fragrance and flavor compositions. The presently disclosed subject matter addresses these needs through compounds disclosed herein and/or new fragrance and flavor compositions comprising one or more of the disclosed compounds.

For clarity, and not by way of limitations, the detailed description is divided into the following subsections:

1. Definitions;

2. Derivatives of 1-(2-hydroxy-4-methylcyclohexyl)-ethanone;

3. Synthesis of Derivatives of 1-(2-hydroxy-4-methylcyclohexyl)-ethanone;

4. Fragrance Compositions;

5. Use of Fragrance Compositions in Consumer Products;

6. Flavor Compositions;

7. Use of Flavor Compositions in Consumer Products.

### 1. Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compositions and methods of the disclosure and how to make and use them.

As used herein, the use of the word “a” or “an” when used in conjunction with the term “comprising” in the claims and/or the specification can mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents, or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Therefore, the presently disclosed subject matter includes enantiomers, diastereomers, or racemates of the compound. Also, as used herein, the terms “constitutional isomers” refers to different compounds which have the same numbers of, and types of, atoms but the atoms are connected differently.

As used herein, the term “fragrance composition” refers to a mixture comprising one or more fragrance components, in any of their forms, and one or more solvents or perfuming co-ingredients. As known in the art, a fragrance composition contains one or more fragrance components (e.g., perfuming co-ingredients) in order to impart an olfactory note to the composition (e.g., a household cleaner, perfume, or other consumer product) to which it is added. In one embodiment, the fragrance composition contains two or more fragrance components which, collectively and in combination with the solvent to which they are added, impart an intended olfactory note (e.g., a hedonically pleasing “tropical” note) to a human in close proximity to the fragrance composition.

In general terms, perfuming co-ingredients belong to chemical classes as varied as alcohols, aldehydes, ketones, esters, ethers, acetates, nitriles, terpene hydrocarbons, nitrogenous or sulphurous heterocyclic compounds and essential oils of natural or synthetic origin, and are known to perfumists of ordinary skill in the art. Many of these ingredients are listed in reference texts such as S. Arctander, Perfume and Flavor Chemicals, 1969, Montclair, N.J., USA or any of its more recent versions, each of which are hereby incorporated by reference.

As used herein, the term “flavor composition” refers to a composition that contains one or more compounds (e.g., co-ingredients) that provide a desired taste when combined with a solvent that is suitable for oral administration and oral consumption. Examples of flavoring co-ingredients that are generally included in a flavor composition are listed in S. Arctander, Perfume and Flavor Chemicals, 1969, Montclair, N.J., USA. The skilled person in the art of flavors is able to select them on the basis of its general knowledge and according to the nature of the product to be flavored and the desired taste.

As used herein, the phrase “consumer product” or “end product” refers to composition that is in a form ready for use by the consumer for the marketed indication. A solvent suitable for use in a consumer product is a solvent that, when combined with other components of the end product, will not render the consumer product unfit for its intended consumer use.

### 2. Derivatives of 1-(2-Hydroxy-4-Methylcyclohexyl)-Ethanone

The compound, 1-(2-hydroxy-4-methylcyclohexyl)-ethanone, can be prepared by methods known to those skilled in the art, such as those described in U.S. Pat. No. 8,071,531 and PCT Patent Application Publication No. WO2017/044957, both of which are incorporated herein by reference in their entirety. Starting from 1-(2-hydroxy-4-methylcyclohexyl)-ethanone, novel fragrance and flavor compounds can be derived, i.e., alcohols, ethers, esters, ketones, acetals, ketals, and oximes of 1-(2-hydroxy-4-methylcyclohexyl)-ethanone.

In certain embodiments, the presently disclosed subject matter provides derivative compounds that are alcohols. In certain embodiments, the presently disclosed subject matter provides derivative compounds that are ethers. In certain embodiments, the presently disclosed subject matter provides derivative compounds that are esters. In certain embodiments, the presently disclosed subject matter provides derivative compounds that are ketones. In certain embodiments, the presently disclosed subject matter provides derivative compounds that are ketals. In certain embodiments, the presently disclosed subject matter provides derivative compounds that are oximes.

As depicted in the chemical formulas provided herein, a double line composed of a solid line and a dotted line represents a double bond or a single bond.

In certain embodiments, the presently disclosed subject matter provides the compound of Formula I below:

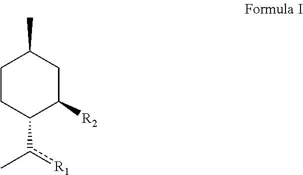

wherein R₁ is one of O, —OH, —NOH—NOR, or NOC(O)R;
wherein R₂ is one of —OC(O)R, —OH, or —OTHP; and
wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

In certain embodiments, the present disclosure provides compound of Formula I, wherein R₁ is one of —OH, —NOH—NOR, or NOC(O)R; and wherein R₂ is —OH.

In certain embodiments, the presently disclosed subject matter provides the compound represented by Formula Ia:

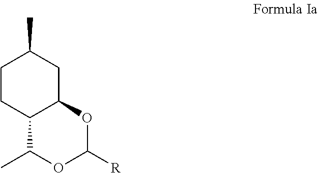

wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

In certain embodiments, the presently disclosed subject matter provides the compound represented by Formula Ib:

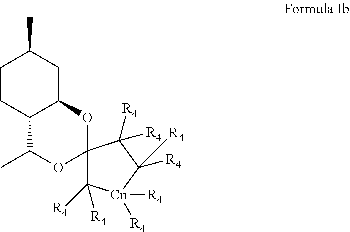

wherein n is 1, 2, 3, or 4;
wherein each R₄ is independently H or C₁-C₈ alkyl.

In certain embodiments, the presently disclosed subject matter provides the compound of Formula II below:

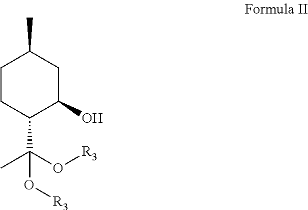

wherein each R₃ is independently H, C₁-C₈ alkyl, C₁-C₈ branched alkyl, C₁-C₈ alkenyl or C₁-C₈ branched alkenyl.

In certain embodiments, the presently disclosed subject matter provides the compound represented by Formula IIa:

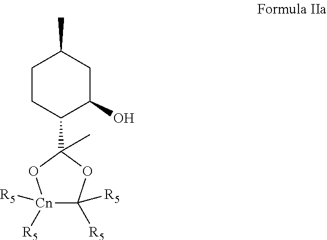

wherein n is 1 or 2;

wherein each R₅ is independently H, C₁-C₈ alkyl, C₁-C₈ branched alkyl, C₁-C₈ alkenyl or C₁-C₈ branched alkenyl.

In a particular embodiment of the present disclosure, the compound can be (7R)-4,7-dimethylhexahydro-4H-spiro[benzo[d][1,3]dioxine-2,1′-cyclohexane].

In a different embodiment of the present disclosure, the compound can be (7R)-4,7-dimethyl-2-(pentan-2-yl)hexahydro-4H-benzo[d][1,3]dioxine.

In another embodiment of the present disclosure, the compound can be (7R)-2-isopropyl-4,7-dimethylhexahydro-4H-benzo[d][1,3]dioxine.

In particular embodiments of the present disclosure, the compound can be (1R,2R,5R)-2-acetyl-5-methylcyclohexyl propionate, (1R,2R,5R)-2-acetyl-5-methylcyclohexyl butyrate, (1R,2R,5R)-2-acetyl-5-methylcyclohexyl isobutyrate, (1R,2R,5R)-2-acetyl-5-methylcyclohexyl pentanoate, (1R,2R,5R)-2-acetyl-5-methylcyclohexyl cyclopropanecarboxylate, (1R,2R,5R)-2-acetyl-5-methylcyclohexyl cyclohexanecarboxylate, (1R,2R,5R)-2-acetyl-5-methylcyclohexyl 2-methylbutanoate, (1R,2R,5R)-2-acetyl-5-methylcyclohexyl methacrylate, (1R,2R,5R)-2-acetyl-5-methylcyclohexyl cyclopentanecarboxylate, (1R,2R,5R)-2-acetyl-5-methylcyclohexyl benzoate, (1R,2R,5R)-5-methyl-2-(2-methyl-1,3-dioxolan-2-yl)cyclohexan-1-ol, (1R,2R,5R)-5-methyl-2-(2,5,5-trimethyl-1,3-dioxan-2-yl)cyclohexan-1-ol, (1R,2R,5R)-2-((2R)-4-ethyl-2-methyl-1,3-dioxolan-2-yl)-5-methylcyclohexan-1-ol, 1-((1R,2R,4R)-2-hydroxy-4-methylcyclohexyl)ethan-1-one oxime, 1-((1R,2R,4R)-2-hydroxy-4-methylcyclohexyl)ethan-1-one O-methyl oxime, (1R,2S,5R)-2-((S)-1-hydroxyethyl)-5-methylcyclohexan-1-ol, (7R)-2-(tert-butyl)-4,7-dimethylhexahydro-4H-benzo[d][1,3]dioxine, (7R)-2-ethyl-4,7-dimethylhexahydro-4H-benzo[d][1,3]dioxine, or 1-((1R,2R,4R)-4-methyl-2-((tetrahydro-2H-pyran-2-yl)oxy)cyclohexyl)ethan-1-one.

### 3. Synthesis of Derivatives of 1-(2-hydroxy-4-methylcyclohexyl)-ethanone

The presently disclosed compounds can be prepared synthetically through various chemical transformations. The Examples provided here describe various nonlimiting embodiments of synthetic procedures. The compounds that are alcohols can be prepared using a reducing agent such as sodium borohydride or lithium aluminum hydride. The compounds that are ethers can be prepared using an alkyl halide, such as methyl iodide, in the presence of a strong base, such as sodium hydride. The compounds that are esters can be prepared using an anhydride, such as acetic anhydride. The compounds that are ketones can be prepared using an oxidizing agent such as sodium hypochlorite or pyridinium chlorochromate. The compounds that are acetals and ketals can be prepared using a diol, such as ethylene glycol or propylene glycol, in the presence of a weak organic acid such as propionic acid. The compounds that are oximes can be prepared using a hydroxy amine salt, such as hydroxylamine hydrochloride.

### 4. Fragrance Compositions

In certain embodiments, any one of the above described compounds can be provided in a fragrance composition. Certain embodiments of the presently disclosed subject matter provide a method to modify, enhance or improve the odor properties of a fragrance composition by adding to the composition an effective quantity of one or more of the compounds.

For fragrance applications, concentrations of the compounds of Formulas (I-II) are based on the total weight of the composition into which the fragrance compound is incorporated. For fragrance applications, typical concentrations of the presently disclosed compounds range from about 0.001% to about 20% by weight, or from about 0.01% to about 10% by weight, or from about 0.1% to about 5%, or from about 1% to about 5%, based on the total weight of the composition into which the fragrance compound is incorporated. Those skilled in the art are able to employ the desired level of the compounds of the disclosed subject matter to provide the desired fragrance/flavor and intensity.

The compounds of the presently disclosed subject matter can be combined with one or more fragrance accords or compounds from various fragrance categories including but not limited to one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

Non-limiting examples of suitable aldehydic compounds include acetaldehyde C-8, acetaldehyde C-9, acetaldehyde C-10, adoxal, aldehyde C-8, aldehyde C-9, aldehyde C-10, aldehyde C-11, aldehyde C-12, aldehyde C-12 lauric, aldehyde C-12 MNA, aldehyde supra, cyclomyral trans-2-decenal, trans-4-decenal, cis-4-decenal, 9-decenal, myrac aldehyde, precyclemone B, trans-2-dodecenal, undecylenic aldehyde, 1-methyl-4-(4-methylpentyl)cyclohex-3-ene-1-carbaldehyde (VERNALDEHYDE®), and combinations thereof.

Non-limiting examples of an animalic compound are 5-Cyclohexadecen-1-one (AMBRETONE®), 17-oxacycloheptadec-6-en-1-one (ambrettolide), 2,5,5-trimethyl-1,3,4,4a,6,7-hexahydronaphthalen-2-ol (ambrinol), 2-Methyl-5-(5,5,6-trimethylbicyclo[2.2.1]hept-2-yl)cyclohexanone (ALDRON®), civet, p-cresol, cresyl methyl ether, indole, skatole, and combinations thereof.

Non-limiting examples of a balsamic compound are benzy salicylate, cylohexyl salicylate, isopropoxy ethyl salicylate, phenethyl salicylate, styrax oil, and combinations thereof.

Non-limiting examples of a citrus compound are delta-3-carene, citral, citronellal, L-cintronellol, decanal, DH-L-citronellol, myrcenol, limonene, DH-myrcenol, nootkatone, sinensal, rhubafuran, bergamot oil, grapefruit oil, lemon oil, lime oil, orange oil, mandarin oil, tridecene-2-nitrile, and/or yuzu core base.

Non-limiting examples of a floral compound are acetanisole, alpha amyl cinnamaldehyde, anisyl acetate, anisic aldehyde, benzyl acetate, bourgeonal, butyl acetate, 1-citrol, cyclamen aldehyde, cyclohexyl lactone, delta-damascone, 9-decen-1-ol, dimethyl benzyl carbinol, farnesal,1-dihydrofarnesal, ethyl linalool,1-farnesal, farnesol, 1-dihydrofarnesol, 3-(3-Isopropylphenyl)butanal (FLORHYDRAL®), 3-(4-ethylphenyl)-2,2-dimethylpropanal (floralozone), 4-methyl-2-(2-methylpropyl)oxan-4-ol (FLOROL®), geraniol, geranyl acetate, piperonal, methyl 3-oxo-2-pentylcyclopentaneacetate (Hedione®), 2-Methyl-3-(3,4-methylenedioxyphenyl)propanal (Heliobouquet), hexyl cinnamaldehyde, hexyl salicylate, indole, alpha-ionone, beta-ionone, isopropoxy ethyl salicylate, methyl-2-((1S*,2R*)-3-oxo-2-pentylcyclopentyl)acetate (JASMODIONE®), cis-jasmone, 4-(4-hydroxy-4-methylpentyl)cyclohex-3-ene-1-carbaldehyde (KOVANOL®), laurinal, lilial, linalool, linalyl acetate, 2,4,6,-trimethyl-4-phenyl-1,3-dioxane (LOREXAN®), 2,4-Dimethyl-4,4a,5,9b-tetrahydroindeno[1,2-d][1,3]dioxine (Magnolan), (4-propan-2-ylcyclohexyl)methanol (Mayol), methyl dihydrojasomante, gamma-methyl ionone, methoxymelonal, methyl benzoate, 1-(4-Isopropyl-cyclohexyl) ethanol (Mugetanol), nerol, 1-(3-Methyl-benzofuran-2-yl)-ethanone (Nerolione), neryl acetate, neroli oil, orantha, L., 2-pentyl cyclopentanone, 2-cyclohexyl-2-cyclohexylideneacetonitrile (PEONILE®), phenoxanol, phenoxy ethyl isobutyrate, phenylacetaldehyde, phenyl ethyl alcohol, prenyl salicylate, rose oxide, rosephenone, rosyrane, suzaral, terpineol, undecavertol, 2,2,5-trimethyl-5-pentylcyclopentan-1-one (VELOUTONE®), yara yara, geranium oil, rose oil, lavender oil, ylang oil, and combinations thereof.

Non-limiting examples of a fruity compound are aldehyde C-16, allyl caproate, allyl cyclohexyl proprionate, allyl heptanoate, amyl acetate, benzaldehyde, cassis oil, L-citronellyl acetate, L-citronellyl nitrile, 3a,4,5,6,7,7a-hexahydro-4,7-methano-1h-inden-5 (or 6)-yl acetate (CYCLACET®), 3a,4,5,6,7,7a-hexahydro-1H-4,7-methanoinden-1-yl propanoate (CYCLAPROP®), cyclogalbanate, damascenone, beta-decalactone, gamma-decalactone, diethyl malonate, dimethyl benzyl carbinol acetate, dimethyl benzyl carbinyl butyrate, dimethyl phenyl ethyl carbinol, dimethyl sulfide, γ-dodecalactone, ethyl acetate, ethyl butyrate, ethyl caproate, ethyl decadienotate, ethyl heptoate, ethyl-2-methylbutyrate, ethyl acetoacetate, ethyl methyl phenyl glycidate, ethyl propionate, 4-methyl-2-(2-methylpropyl)oxan-4-ol (FLOROL®), ethyl tricyclo [5.2.1.02.6] decan-2 carboxylate (FRUITATE®), hexyl acetate, hexyl isobutyrate, isoamyl acetate, 6-(pent-3-en-1-yl)tetrahydro-2H-pyran-2-one (Jasmolactone), ethyl 2-methylpentanoate (manzanate), 2,6-dimethylhept-5-enal (melonal), methyl anthranilate, methyl dioxolan, methyl heptyl ketone, gamma-nonalactone, 6-nonenol, gamma-octalactone, phenyl ethyl isobutyrate, prenyl acetate, raspberry ketone, methyl(2-((4S)-4-methyl-2-methylenecyclohexyl)propan-2-yl)sulfane (RINGONOL®), (1R, 6S)-ethyl 2,2,6-trimethylclocloxehanecarboxylate (THESARON®), tolyl aldehyde, γ-undecalactone, 3,5,5-trimethylhexyl acetate (vanoris), (2-tert-butylcyclohexyl) acetate (verdox), and combinations thereof.

Non-limiting examples of a gourmand compound are angelica lactone-alpha, caprylic acid, coumarin, ethyl fraison, ethyl vanillin, ethyl maltol (e.g., VELTOL PLUS), filbertone, 4-hydroxy-2,5-dimethyl-3 (2H)-furanone (FURANEOL®), guaiacol, maple furanone, 2-acetyl pyrazine, 2,5-dimethyl pyrazine, vanillin and combinations thereof.

Non-limiting examples of a green compound are allyl amyl glycolate, cyclogalbanate, 1-(5,5-dimethyl-1-cyclohexen-1-yl)-4-Penten-1-one (DYNASCONE®), galbanolene, galbanum, trans-2-hexenal, cis-3-hexenol, hexen-1-ol, cis-3-hexenyl acetate, cis-3-hexenyl butyrate, cis-3-hexenyl formate, cis-3-hexenyl salicyclate, liffarome, 2-methoxy-2-methylheptane, methyl octine carbonate, neofolione, 2,6-nonadienal, (2R,4S)-2-methyl-4-propyl-1,3-oxathiane (OXANE®), octahydro-5-methoxy-4,7-methano-1H-indene-2-carboxaldehyde (SCENTENAL®), N-(5-methylheptan-3-ylidene)hydroxylamine (STEMONE®), styrallyl acetate, 2,4-dimethylcyclohex-3-ene-1-carbaldehyde (TRIPLAL®), undecavertol, vionil, violet methyl carbonate (e.g., VIOLET T), violet leaf extract, and combinations thereof.

Non-limiting examples of an herbaceous compound are anethol, bamboo ketone, canthoxal, carvacrol, carvone, l., clary sage natural oil, cymene, p., Daikon Ether, 2,6-dimethylheptan-2-ol (DIMETOL®), menthol, methyl salicylate, thymol, natural basil oil, natural eucalyptus oil, eucalyptol, sweet natural fennel oil, natural cedar leaf oil, and combinations thereof.

Non-limiting examples of a marine compound are 8-methyl-1,5-benzodioxepin-3-one (Calone® 1951), 3-(4-ethylphenyl)-2,2-dimethylpropanal (floralozone), 4-tert-butylphenylacetonitrile (MARENTL®), 4-[(3E)-4,8-dimethylnona-3,7-dienyl]pyridine (MARITIMA®), myrac aldehyde, ultrazure, and combinations thereof.

Non-limiting examples of a mossy compound are hinokitiol, isobutyl quinolone, isopropyl quinolone and/or methyl 2,4-dihydroxy-3,6-dimethylbenzoate (Oakmoss™ #1), and combinations thereof.

Non-limiting examples of a musk compound are 17-oxacycloheptadec-6-en-1-one (ambrettolide), 5-Cyclohexadecen-1-one (AMBRETONE®), (3aR,5aS,9aS,9bR)-3a,6,6,9a-Tetramethyldodecahydronaphtho[2,1-b]furan (AMBROXAN), 2,2,6-trimethyl-alpha-propylcyclohexanepropanol (Dextramber), 16-oxacyclohexadecan-1-one (EXALTOLIDE®), galaxolide, ((12E)-1-oxacyclohexadec-12-en-2-one (habanolide), [2-[1-(3,3-dimethylcyclohexyl)ethoxy]-2-methylpropyl]propanoate (HELVETOLIDE®), (1′R)-3-methyl-5-(2,2,3-trimethylcyclopentan-1-yl)-2-pentanone, (5E)-3-methylcyclopentadec-5-en-1-one (MUSCENONE®), 1,4-dioxacycloheptadecane-5,17-dione (Musk T), 3-methylcyclopentadecan-1-one (L-muscone), 1-(3,5,5,6,8,8-hexamethyl-6,7-dihydronaphthalen-2-yl)ethenone (TONALID®), and combinations thereof.

Non-limiting examples of a piney compound are 1-borneol, 1-bornyl acetate, cypress oil, camphene, camphor gum powder, dihydroterpineol, β-pinene, and combinations thereof.

Examples of a powdery compound include, without limitation, heliotropine and/or whiskey lactone (methyl octalactone).

Non-limiting examples of a spicy compound are acetyl isoeugenol, delta-caryophellene, cardamom oil, cinnamaldehyde, cuminaldehyde, eugenol, isoeugenol, perilla aldehyde, cardamom oil, clove oil, ginger extract, ginger oil, black pepper extract and combinations thereof.

Non-limiting examples of a woody and/or amber compound are amber core, amber extreme, ambretol, 4aR,5R,7aS,9R)-Octahydro-2,2,5,8,8,9a-hexamethyl-4H-4a,9-methanoazuleno[5,6-d]-1,3-dioxole (AMBROCENIDE®), ((3aR,5aS,9aS,9bR)-3a,6,6,9a-Tetramethyldodecahydronaphtho[2,1-b]furan (AMBROXAN), 2-ethyl-4-(2,2,3-trimethyl-3-cyclo-penten-1-yl)-2-buten-1-ol (BACDANOL®), ethoxymethoxycyclododecane (Boisambrene Forte), 1,1,2,3,3-pentamethyl-2,5,6,7-tetrahydroinden-4-one (Cashmeran®), ((2R,5S,7R,8R)-8-methoxy-2,6,6,8-tetramethyltricyclo[5.3.1.01,5]undecane (Cedramber®), cedanol, cedarwood oil, (1S,2R,5S,7R,8R)-2,6,6,8-tetramethyltricyclo[5.3.1.01,5]undecan-8-ol (Cedrol), 2,2,6-trimethyl-alpha-propylcyclohexanepropanol (Dextramber), 3-Methyl-5-(2,2,3-trimethyl-3-cyclopenten-1-yl)-4-penten-2-ol (EBANOL®), (R,E)-2-Methyl-4-(2,2,3-trimethylcyclopent-3-enyl) but-2-en-1-ol (HINDINOL®), hinokitiol, DH-ionone beta, [(1R,2S)-1-methyl-2-[[(1R,3S,5S)-1,2,2-trimethyl-3-bicyclo[3.1.0]hexanyl]methyl]cyclopropyl]methanol (JAVANOL®), 5-butan-2-yl-2-(2,4-dimethyl-1-cyclohex-3-enyl)-5-methyl-1,3-dioxane (karanal), 2,4-Dimethyl-2-(1,1,4,4-tetramethyltetralin-6-yl)-1,3-dioxolane (OKOUMAL®), 1-(1,2,3,4,5,6,7,8-octahydro-2,3,8,8-tatramethyl-2-naphthyl)ethan-1-one (ORBITONE®), patchouly oil, polysantol, rhubofix, sandalwood, and combinations thereof.

The amounts of the fragrance compounds can vary depending on the intended resulting fragrance composition, but can range from about 0.1 parts per thousand to about 800 parts per thousand, or from about 1 part per thousand to about 500 parts per thousand.

Such compositions can contain or consist of at least one ingredient selected from a group consisting of a fragrance carrier and a fragrance base. Such compositions can also consist of at least one fragrance adjuvant.

Fragrance carriers can be a liquid or a solid and typically do not significantly alter the olfactory properties of the fragrance ingredients. Some non-limiting examples of fragrance carriers include an emulsifying system, encapsulating materials, natural or modified starches, polymers, gums, pectins, gelatinous or porous cellular materials, waxes, and solvents which are typically employed in fragrance applications.

Fragrance base refers to any composition comprising at least one fragrance co-ingredient. In general, these co-ingredients belong to chemical classes such as, but not limited to: alcohols, aldehydes, ketones, esters, ethers, acetals, oximes, acetates, nitriles, terpenes, saturated and unsaturated hydrocarbons, and essential oils of natural or synthetic origins.

The fragrance compositions according to the disclosed subject matter can be in the form of a simple mixture of the various co-ingredients and solvents, or also in the form of a biphasic system such as an emulsion or microemulsion. Such systems are well-known to persons skilled in the art.

Nonlimiting examples of such solvents used in perfumery are known in the art and include but are not limited to: dipropyleneglycol, diethyl phthalate, isopropyl myristate, benzyl benzoate, 2-(2-ethoxy)-1-ethanol, ethyl citrate, ethanol, water/ethanol mixtures, limonene or other terpenes, isoparaffins such as those known under the trademark Isopar® (ExxonMobil Chemicals, Houston, Tex.), and glycol ethers and glycol ether esters such as those known under the trademark Dowanol® (Dow Chemical Company, Midland Mich.).

### 5. Use of Fragrance Compositions in Consumer Products

The fragrance compositions of the presently disclosed subject matter as described above can be advantageously used within a wide variety of consumer products. Suitable end products that can include a compound of the presently disclosed subject matter include, but are not limited to: 1) candles, air fresheners, perfumes, and colognes; 2) personal care products such as soaps, deodorants, shampoos, conditioners, shower gels, and shaving lotions; 3) cosmetics such as lotions and ointments; and 4) detergents, fabric care products, and household cleansers/cleaning agents. Depending on the solvents that can be present in some end products, it can be necessary to protect the compounds from premature degradation, for example by encapsulation or with a stabilizer, or other methods well-known to those of ordinary skill in the art.

The above-listing of end products is non-limiting. The compositions of the presently disclosed subject matter can be included in a number of additional products, including for example: 1) fragrance products, eau de perfume, eau de toilet, eau de cologne, and the like; skin-care cosmetics, face washing creams, varnishing creams, cleansing creams, cold creams, massage creams and oils, milky lotions, skin toning lotion, cosmetic solutions, packs, makeup remover, and the like; 2) makeup cosmetics, foundations, face powders, pressed powders, talcum powders, lip sticks, lip creams, cheek powders, eyeliners, mascara, eye shadows, eyebrow pencils, eye packs, nail enamels, nail enamel removers, and the like; 3) hair care cosmetics, pomades, brilliantines, setting lotions, hair sticks, hair solids, hair oils, hair treatments, hair creams, hair tonics, hair liquids, hair sprays, hair restorers, hair dyes, and the like; 4) sunburn cosmetics, suntan products, sunscreen products, and the like; 5) medical cosmetics, antiperspirants, after-shave lotions and gels, permanent wave lotions, medicated soaps, medicated shampoos, medicated skin care products, and the like; 6) hair care products, rinses, shampoo—including-rinses, hair conditioners, hair treatments, hair packs, and the like; 7) as bath soaps, perfumed soaps, transparent soaps, synthetic soaps, and the like; 8) body washing soaps, body soaps, body shampoos, hand soaps, and the like; 9) bathing, bathing agents (e.g., bath salts, bath tablets, bath liquids, and the like), foam baths (bubble bath and the like), bath oils (e.g., bath perfumes, bath capsules, and the like), milk baths, bath gels, bath cubes, and the like; 10) heavy duty detergents for clothes, light duty detergents for clothes, liquid detergents, laundering soaps, compact detergents, powder soaps, and the like; 11) softening finishing agents, softeners, furniture care products, and the like; deodorants, aromatic substances, and the like; 12) insect repellent, insecticides, and the like; 13) oral care products such as tooth pastes, mouth cleaners, mouth wash, troches, chewing gums, and the like; and 14) pharmaceutical products, poultices, external skin care pharmaceuticals such as ointments, internal administration medicines, and the like.

### 6. Flavor Compositions

In certain embodiments, any one of the above-described compounds, alone or in combination, can be provided in a flavor composition. Certain embodiments of the presently disclosed subject matter provide a method to modify, enhance, or improve the taste properties of a flavor composition by adding to the composition an effective quantity of one or more of the presently disclosed compounds.

For flavor applications, concentrations of the presently disclosed compounds are based on the total weight of the composition into which the flavor compound is incorporated. For flavor applications, typical concentrations of the compounds can range from about 0.0001% to about 20% by weight, or from about 0.01% to about 10% by weight, or from about 0.1% to about 5%, or from about 0.1% to about 5%, based on the total weight of the composition into which the compound is incorporated. Those skilled in the art are able to employ the desired level of said compounds to provide the desired flavor and intensity. Much higher concentrations can be employed when the compounds are used in concentrated flavors and flavor compositions.

As used herein, organoleptic effective quantity will be defined as the amount of said compound in a flavor composition in which the individual component will contribute its characteristic flavor properties. However, the organoleptic effect of the flavor composition will be the sum of the effects of all flavor ingredients present. Therefore, the compounds embodied in the presently disclosed subject matter can be employed to modify the characteristics of the flavor composition via their own organoleptic properties or through additively effecting the contributions of other ingredient(s) present within the said composition. The quantity will vary widely depending on the presence of other ingredients present, their relative amounts, the desired effect, and the nature of the product.

The flavor carrier can be a liquid or a solid, and typically does not significantly alter the olfactory or organoleptic properties of the flavor ingredients, respectively. Some non-limiting examples of flavor carriers include an emulsifying system, encapsulating materials, natural or modified starches, polymers, pectins, proteins, polysaccharides, gums and solvents which are typically employed in flavor applications.

The flavor compositions according to the disclosed subject matter can be in the form of a simple mixture of the various co-ingredients, adjuvants, and solvents, or also in the form of a biphasic system such as an emulsion or microemulsion. Such systems are well-known to persons skilled in the art.

Nonlimiting examples of solvents commonly used in flavors are also known in the art and include, but are not limited to: water, medium-chain triglycerides (MCTs), propylene glycol, triacetin, triethyl citrate, benzyl alcohol, benzyl benzoate, ethanol, vegetable oils, and terpenes.

As used herein, the term “flavor carrier” can also encompass the food, confectionaries, beverage, oral care product, or pharmaceutical to which the flavor compound (i.e., compounds of the present disclosure) is added. Examples of such foods, confectionaries, beverages, oral care products, or pharmaceuticals include, but are not limited to carbonated fruit beverages, carbonated cola drinks, wine coolers, cordials, flavored water, powders for drinks (e.g., powdered sports or “hydrating” drinks), hard candy, soft candy, taffy, chocolates, sugarless candies, chewing gum, bubble gum, alcoholic beverages, energy beverages, juices, teas, coffees, breath freshener tablets or drops, film strips for halitosis, gelatin candies, pectin candies, starch candies, lozenges, cough drops, throat lozenges, throat sprays, toothpastes, and mouth rinses.

### 7. Use of Flavor Compositions in Consumer Products

The flavor compositions of the presently disclosed subject matter as described above can be advantageously used within a wide variety of consumer products intended to be eaten, imbibed or otherwise consumed. Suitable end products that can include a compound of the presently disclosed subject matter include but are not limited to: beverages such as juices, sodas, teas, flavored waters, fruit-based “smoothie” drinks, milk-based drinks, and the like; confectionaries such as sweets, hard candy, gums; and pharmaceuticals, oral care products, and the like.

The flavor compositions according to the disclosed subject matter can be in the form of a simple mixture of flavoring ingredients or in an encapsulated form, e.g., a flavoring composition entrapped into a solid matrix that can comprise wall-forming and plasticizing materials such as mono-, di-, or trisaccharides, natural or modified starches, hydrocolloids, cellulose derivatives, polyvinyl acetates, polyvinylalcohols, proteins, or pectins. Examples of particularly useful matrix materials include, for example, sucrose, glucose, lactose, levulose, fructose, maltose, ribose, dextrose, isomalt, sorbitol, mannitol, xylitol, lactitol, maltitol, pentatol, arabinose, pentose, xylose, galactose, maltodextrin, dextrin, chemically modified starch, hydrogenated starch hydrolysate, succinylated or hydrolysed starch, agar, carrageenan, gum arabic, gum accacia, tragacanth, alginates, methyl cellulose, carboxymethyl cellulose, hydroxyethyl cellulose, hydroxypropylmethyl cellulose, derivatives, gelatin, agar, alginate, and mixtures thereof. Encapsulation is well-known to persons skilled in the art, and can be performed, for instance, using techniques such as spray-drying, agglomeration or extrusion, or coating encapsulation, including coacervation and complex coacervation techniques.

In one embodiment, a compound of the presently disclosed subject matter is included/used in chewing and bubble gums and confectionaries (e.g., hard or soft candies or lozenges). Chewing gum compositions typically include one or more gum bases and other standard components such as flavoring agents, softeners, sweeteners, and the like. Flavoring agents for use in chewing gum compositions are well known and include natural flavors such as citrus oils, peppermint oil, spearmint oil, oil of wintergreen, natural menthol, cinnamon, ginger, and the like; and artificial flavors such as menthol, carvone, limonene, cinnamic aldehyde, linalool, geraniol, ethyl butyrate, and the like. As is known in the art, the ingredients used in chewing gum compositions can include sweeteners, both natural and artificial and both sugar and sugarless. Sweeteners are typically present in the chewing gum compositions in amounts of from about 20% to 80% by weight, or from about 30% to 60% by weight, based on the total weight of the chewing gum composition. Sugarless sweeteners include, but are not limited sugar alcohols such as Sorbitol, manifold, xylitol, hydrogenated starch hydrolysates, malitol, and the like. High intensity sweeteners such as sucralose, aspartame, neotame, salts of acesulfame, and the like, when employed, are typically present up to about 1.0% by weight.

In an alternative embodiment, a compound of the presently disclosed subject matter is included in an oral personal care product (e.g., a mouthwash or toothpaste). For example, a mouthwash can be prepared by dissolving a flavor composition (e.g., a flavor cocktail) (liquid or powder) that includes a compound of the presently disclosed subject matter in a solvent (e.g., water) that further includes, for example, a flavor such as menthol and a surfactant; and then mixing the resulting solution with, for example, an aqueous erythritol solution.

In one embodiment of the presently disclosed subject matter, a compound of the presently disclosed subject matter is added, directly or indirectly, to a pharmaceutical dosage form (e.g., a tablet, capsule, drop, or lozenge) that contains a therapeutically active agent (e.g., a medicament). For example, one embodiment of the presently disclosed subject matter provides a cough drop or lozenge containing one or more compounds of the present disclosure and, optionally, further containing menthol or other medicaments for the treatment of sore throat, coughing or other upper respiratory ailments.

### EXAMPLES

The present application is further described by means of the examples, presented below, wherein the abbreviations have the usual meaning in the art.

The use of such examples is illustrative only and does not limit the scope and meaning of the disclosed subject matter or of any exemplified term. Likewise, the disclosed subject matter is not limited to any particular preferred embodiments described herein. Indeed, many modifications and variations of the disclosed subject matter are apparent to those skilled in the art upon reading this specification. The disclosed subject matter is therefore to be limited only by the terms of the appended claims along with the full scope of equivalents to which the claims are entitled.

The following Examples are provided as specific embodiments of the present disclosure, wherein the abbreviations have the usual meaning in the art. The temperatures are indicated in degrees centigrade (° C.); the NMR spectral data were recorded in CDCl₃ with a 400 MHz machine for ¹H and ¹³C, the chemical displacements are indicated in ppm with respect to TMS as the standard. The reaction progress was monitored by gas chromatography (GC).

### Example 1: Synthesis of (1R,2R,5R)-2-acetyl-5-methylcyclohexyl propionate

(1R,2R,5R)-2-acetyl-5-methylcyclohexyl propionate was prepared and characterized as follows.

To a 2-neck round bottom flask, equipped with a thermometer, was added 1-((1R,2R,4R)-2-hydroxy-4-methylcyclohexyl)ethan-1-one (2.0 g, 12.8 mmol) and anhydrous dichloromethane (50 mL). Pyridine (19.2 mmol) was added and the stirring mixture was cooled to 0° C. Propionyl chloride (14.1 mmol) was added dropwise, keeping the internal temperature under 5° C. After addition, let the mixture warmed to room temperature where it remained until completion. The reaction mixture was then diluted with dichloromethane and washed with water (1×), 1N HCl (1×) and brine (1×). The organic phase was dried over magnesium sulfate, filtered and concentrated. The crude product was isolated by automated silica gel chromatography (Biotage Isolera, 5-40% ethyl acetate/hexanes) to afford 2.15 g (79%) of (1R,2R,5R)-2-acetyl-5-methylcyclohexyl propionate. ¹H NMR (400 MHz, CHCl₃-d) δ ppm 0.92 (m, 5H) 1.07 (m, 3H) 1.38 (dd, J=13.28, 3.66 Hz, 1H) 1.55 (td, J=6.30, 2.98 Hz, 1H) 1.72 (m, 1H) 1.88 (m, 1H) 2.11 (m, 4H) 2.22 (m, 2H) 2.54 (ddd, J=12.59, 10.76, 4.12 Hz, 1H) 4.95 (td, J=10.99, 4.58 Hz, 1H). ¹³C NMR (101 MHz, CHCl₃-d) δ ppm 9.20, 21.90, 27.79, 27.85, 29.12, 30.92, 33.47, 39.54, 55.70, 73.16, 173.56, 210.01

### Example 2: Synthesis of (1R,2R,5R)-2-acetyl-5-methylcyclohexyl butyrate

(1R,2R,5R)-2-acetyl-5-methylcyclohexyl butyrate was prepared and characterized as follows.

The same procedure shown in Example 1 was followed to synthesize (1R,2R,5R)-2-acetyl-5-methylcyclohexyl butyrate using butyryl chloride as a substrate. The yield for this reaction was 70%. ¹H NMR (400 MHz, CHCl₃-d) δ ppm 0.91 (m, 8H) 1.37 (dd, J=13.28, 3.66 Hz, 1H) 1.57 (m, 3H) 1.70 (m, 1H) 1.88 (m, 1H) 2.12 (m, 6H) 2.53 (ddd, J=12.59, 10.76, 4.12 Hz, 1H) 4.95 (td, J=10.99, 4.58 Hz, 1H). ¹³C NMR (101 MHz, CHCl₃-d) δ ppm 13.63, 18.56, 21.89, 27.86, 29.09, 30.92, 33.46, 36.43, 39.55, 55.71, 73.10, 172.76, 209.98

### Example 3: Synthesis of (1R,2R,5R)-2-acetyl-5-methylcyclohexyl isobutyrate

(1R,2R,5R)-2-acetyl-5-methylcyclohexyl isobutyrate was prepared and characterized as follows.

The same procedure shown in Example 1 was followed to synthesize (1R,2R,5R)-2-acetyl-5-methylcyclohexyl isobutyrate using isobutyryl chloride as a substrate. The yield for this reaction was 80%. ¹H NMR (400 MHz, CHCl₃-d) δ ppm 0.92 (m, 5H) 1.08 (m, 6H) 1.38 (dd, J=13.05, 3.43 Hz, 1H) 1.56 (td, J=6.30, 2.98 Hz, 1H) 1.70 (m, 1H) 1.88 (m, 1H) 2.08 (m, 4H) 2.43 (m, 1H) 2.54 (m, 1H) 4.93 (td, J=10.88, 4.35 Hz, 1H). ¹³C NMR (101 MHz, CHCl₃-d) δ ppm 18.90, 19.03, 21.91, 27.87, 29.02, 30.93, 33.48, 34.11, 39.46, 55.77, 73.03, 176.20, 209.97

### Example 4: Synthesis of (1R,2R,5R)-2-acetyl-5-methylcyclohexyl pentanoate

(1R,2R,5R)-2-acetyl-5-methylcyclohexyl pentanoate was prepared and characterized as follows.

The same procedure shown in Example 1 was followed to synthesize (1R,2R,5R)-2-acetyl-5-methylcyclohexyl pentanoate using valeryl chloride as a substrate. The yield for this reaction was 65%. ¹H NMR (400 MHz, CHCl₃-d) δ ppm 0.91 (m, 8H) 1.29 (m, 2H) 1.38 (dd, J=12.82, 3.66 Hz, 1H) 1.55 (m, 4H) 1.71 (m, 1H) 1.89 (dd, J=13.28, 3.21 Hz, 1H) 2.11 (m, 3H) 2.20 (t, J=7.56 Hz, 2H) 2.54 (ddd, J=12.59, 10.76, 3.66 Hz, 1H) 4.95 (td, J=10.99, 4.58 Hz, 1H). ¹³C NMR (101 MHz, CHCl₃-d) δ ppm 13.76, 21.90, 22.24, 27.13, 27.87, 29.09, 30.93, 33.47, 34.27, 39.55, 55.72, 73.11, 172.93, 209.98

### Example 5: Synthesis of (1R,2R,5R)-2-acetyl-5-methylcyclohexyl cyclopropanecarboxylate

(1R,2R,5R)-2-acetyl-5-methylcyclohexyl cyclopropanecarboxylate was prepared and characterized as follows.

The same procedure shown in Example 1 was followed to synthesize (1R,2R,5R)-2-acetyl-5-methylcyclohexyl cyclopropanecarboxylate using cyclopropanecarbonyl acid as a substrate. The yield for this reaction was 69%. ¹H NMR (400 MHz, CHCl₃-d) δ ppm 0.79 (m, 2H) 0.91 (m, 7H) 1.47 (m, 3H) 1.70 (m, 1H) 1.88 (dd, J=13.28, 3.21 Hz, 1H) 2.08 (m, 1H) 2.13 (s, 3H) 2.54 (ddd, J=12.59, 10.76, 4.12 Hz, 1H) 4.94 (td, J=10.99, 4.58 Hz, 1H). ¹³C NMR (101 MHz, CHCl₃-d) δ ppm 8.48, 13.01, 21.89, 27.86, 29.04, 30.94, 33.44, 39.59, 55.79, 73.29, 173.95, 210.07

### Example 6: Synthesis of (1R,2R,5R)-2-acetyl-5-methylcyclohexyl cyclohexanecarboxylate

(1R,2R,5R)-2-acetyl-5-methylcyclohexyl cyclohexanecarboxylate was prepared and characterized as follows.

The same procedure shown in Example 1 was followed to synthesize (1R,2R,5R)-2-acetyl-5-methylcyclohexyl cyclohexanecarboxylate using cyclohexanecarbonyl chloride as a substrate. The yield for this reaction was 74%. ¹H NMR (400 MHz, CHCl₃-d) δ ppm 0.91 (m, 5H) 1.21 (m, 3H) 1.36 (m, 3H) 1.58 (m, 2H) 1.76 (m, 7H) 2.05 (d, J=12.36 Hz, 1H) 2.15 (m, 3H) 2.53 (ddd, J=12.71, 10.65, 3.66 Hz, 1H) 4.93 (td, J=10.88, 4.35 Hz, 1H). ¹³C NMR (101 MHz, CHCl₃-d) δ ppm 21.90, 25.40, 27.88, 28.91, 30.94, 33.47, 39.54, 43.26, 55.88, 72.85, 175.18, 210.00

### Example 7: Synthesis of (1R,2R,5R)-2-acetyl-5-methylcyclohexyl 2-methylbutanoate

(1R,2R,5R)-2-acetyl-5-methylcyclohexyl 2-methylbutanoate was prepared and characterized as follows.

The same procedure shown in Example 1 was followed to synthesize (1R,2R,5R)-2-acetyl-5-methylcyclohexyl 2-methylbutanoate using 2-methylbutyryl chloride as a substrate. The yield for this reaction was 71%. ¹H NMR (400 MHz, CHCl₃-d) δ ppm 0.88 (m, 8H) 1.05 (m, 3H) 1.39 (m, 2H) 1.57 (m, 2H) 1.71 (m, 2H) 1.88 (dd, J=13.28, 3.21 Hz, 1H) 2.10 (m, 4H) 2.24 (m, 1H) 2.54 (m, 1H) 4.94 (m, 1H). ¹³C NMR (101 MHz, CHCl₃-d) δ ppm 11.55, 16.62, 21.90, 26.78, 27.89, 29.00, 30.92, 33.48, 39.53, 41.22, 55.75, 72.99, 175.80, 209.95

### Example 8: Synthesis of (1R,2R,5R)-2-acetyl-5-methylcyclohexyl methacrylate

(1R,2R,5R)-2-acetyl-5-methylcyclohexyl methacrylate was prepared and characterized as follows.

The same procedure shown in Example 1 was followed to synthesize (1R,2R,5R)-2-acetyl-5-methylcyclohexyl methacrylate using methacryloyl chloride as a substrate. The yield for this reaction was 14%. ¹H NMR (400 MHz, CHCl₃-d) δ ppm 0.95 (m, 5H) 1.42 (dd, J=12.82, 3.66 Hz, 1H) 1.60 (m, 2H) 1.73 (ddd, J=13.39, 3.09, 1.83 Hz, 1H) 1.89 (m, 4H) 2.14 (m, 3H) 2.61 (ddd, J=12.71, 10.65, 3.66 Hz, 1H) 5.00 (td, J=10.88, 4.35 Hz, 1H) 5.49 (q, J=1.60 Hz, 1H) 6.00 (m, 1H). ¹³C NMR (101 MHz, CHCl₃-d) δ ppm 18.34, 21.90, 27.84, 29.18, 30.94, 33.45, 39.49, 55.72, 73.85, 125.45, 136.44, 166.45, 210.06

### Example 9: Synthesis of (1R,2R,5R)-2-acetyl-5-methylcyclohexyl cyclopentanecarboxylate

(1R,2R,5R)-2-acetyl-5-methylcyclohexyl cyclopentanecarboxylate was prepared and characterized as follows.

The same procedure shown in Example 1 was followed to synthesize (1R,2R,5R)-2-acetyl-5-methylcyclohexyl cyclopentanecarboxylate using cyclopentanecarbonyl chloride as a substrate. The yield for this reaction was 87%. ¹H NMR (400 MHz, CHCl₃-d) δ ppm 0.91 (m, 5H) 1.38 (dd, J=13.28, 3.66 Hz, 1H) 1.53 (m, 2H) 1.72 (m, 8H) 1.88 (dd, J=13.28, 3.66 Hz, 1H) 2.12 (m, 4H) 2.56 (m, 2H) 4.93 (td, J=10.99, 4.58 Hz, 1H). ¹³C NMR (101 MHz, CHCl₃-d) δ ppm 21.91, 25.86, 27.86, 29.06, 29.84, 30.93, 33.48, 39.51, 43.90, 55.79, 73.04, 175.80, 210.05

### Example 10: Synthesis of (1R,2R,5R)-2-acetyl-5-methylcyclohexyl benzoate

(1R,2R,5R)-2-acetyl-5-methylcyclohexyl benzoate was prepared and characterized as follows.

The same procedure shown in Example 1 was followed to synthesize (1R,2R,5R)-2-acetyl-5-methylcyclohexyl benzoate using benzoyl chloride as a substrate. The yield for this reaction was 63%. ¹H NMR (400 MHz, CHCl₃-d) δ ppm 1.00 (m, 5H) 1.47 (dd, J=12.82, 3.66 Hz, 1H) 1.64 (m, 1H) 1.77 (ddd, J=13.17, 3.09, 1.60 Hz, 1H) 1.95 (dd, J=13.28, 3.21 Hz, 1H) 2.16 (s, 3H) 2.24 (m, 1H) 2.73 (ddd, J=12.59, 10.76, 4.12 Hz, 1H) 5.21 (td, J=10.88, 4.35 Hz, 1H) 7.39 (m, 2H) 7.52 (m, 1H) 7.95 (m, 2H). ¹³C NMR (101 MHz, CHCl₃-d) δ ppm 21.94, 27.93, 29.13, 31.01, 33.48, 39.61, 55.86, 74.13, 128.41, 128.97, 129.59, 130.66, 133.00, 134.63, 165.64, 209.98

### Example 11: Synthesis of (1R,2R,5R)-5-methyl-2-(2-methyl-1,3-dioxolan-2-yl)cyclohexan-1-ol

(1R,2R,5R)-5-methyl-2-(2-methyl-1,3-dioxolan-2-yl)cyclohexan-1-ol was prepared and characterized as follows.

To a one neck, round bottom flask, equipped with a Dean-Stark trap and condenser, was added 1-((1R,2R,4R)-2-hydroxy-4-methylcyclohexyl)ethan-1-one (2.0 g, 12.8 mmol) and toluene (30 mL). While stirring at room temperature, ethylene glycol (64 mmol) was added followed by pyridinium p-toluenesulfonate (1.3 mmol). The mixture was then refluxed until reaction was completed. The reaction mixture was then cooled to room temperature, diluted with ethyl acetate, and quenched with saturated NaHCO₃. Once the phases were separated, the organic phase was washed once more with saturated NaHCO₃ and brine (1×). The organic layer was then dried over magnesium sulfate, filtered, and concentrated under reduced pressure. The crude product was isolated by automated silica gel chromatography (Biotage Isolera, 2-20% ethyl acetate/hexanes) to afford 0.66 g (26%) of (1R,2R,5R)-5-methyl-2-(2-methyl-1,3-dioxolan-2-yl)cyclohexan-1-ol. ¹H NMR (400 MHz, CHCl₃-d) δ ppm 0.88 (m, 5H) 1.30 (s, 3H) 1.44 (m, 2H) 1.65 (m, 2H) 1.82 (m, 1H) 1.98 (m, 1H) 3.58 (td, J=10.30, 4.58 Hz, 1H) 3.96 (m, 4H). ¹³C NMR (101 MHz, CHCl₃-d) δ ppm 20.15, 22.15, 26.55, 31.14, 34.21, 43.44, 50.77, 63.98, 64.66, 70.79, 112.95

### Example 12: Synthesis of (1R,2R,5R)-5-methyl-2-(2,5,5-trimethyl-1,3-dioxan-2-yl)cyclohexan-1-ol

(1R,2R,5R)-5-methyl-2-(2,5,5-trimethyl-1,3-dioxan-2-yl)cyclohexan-1-ol was prepared and characterized as follows.

The same procedure shown in Example 11 was followed to synthesize (1R,2R,5R)-5-methyl-2-(2,5,5-trimethyl-1,3-dioxan-2-yl)cyclohexan-1-ol using 2,2-dimethyl-1,3-propanediol as a substrate. The yield for this reaction was 37%. H NMR (400 MHz, CHCl₃-d) δ ppm 0.85-1.09 (m, 12H) 1.37 (m, 4H) 1.63 (m, 1H) 1.73 (m, 2H) 1.96 (m, 1H) 3.41 (dd, J=11.68, 1.60 Hz, 1H) 3.50 (m, 1H) 3.58 (s, 1H) 3.60 (s, 1H) 3.71 (ddd, J=10.76, 9.62, 4.35 Hz, 1H). ¹³C NMR (101 MHz, CHCl₃-d) δ ppm 16.60, 22.18, 22.56, 22.98, 25.38, 30.20, 31.09, 34.32, 43.57, 49.80, 69.99, 70.21, 70.67, 102.38

### Example 13: Synthesis of (1R,2R,5R)-2-((2R)-4-ethyl-2-methyl-1,3-dioxolan-2-yl)-5-methylcyclohexan-1-ol

(1R,2R,5R)-2-((2R)-4-ethyl-2-methyl-1,3-dioxolan-2-yl)-5-methylcyclohexan-1-ol was prepared and characterized as follows.

The same procedure shown in Example 11 was followed to synthesize (1R,2R,5R)-2-((2R)-4-ethyl-2-methyl-1,3-dioxolan-2-yl)-5-methylcyclohexan-1-ol using 1,2-butanediol as a substrate. The yield for this reaction was 76%. ¹H NMR (400 MHz, CHCl₃-d) δ ppm 0.90 (m, 8H) 1.08 (m, 1H) 1.31 (m, 3H) 1.49 (m, 3H) 1.65 (m, 2H) 1.83 (m, 1H) 1.97 (ddd, J=9.96, 4.69, 2.29 Hz, 1H) 3.54 (m, 2H) 4.06 (m, 2H). ¹³C NMR (101 MHz, CHCl₃-d) δ ppm 9.93, 10.09, 20.33-21.13, 22.16, 26.41-26.57, 31.14, 34.26, 43.47, 50.50, 52.11, 68-49-69.53, 70.63-71.01, 113.00

### Example 14: Synthesis of 1-((1R,2R,4R)-2-hydroxy-4-methylcyclohexyl)ethan-1-one oxime

1-((1R,2R,4R)-2-hydroxy-4-methylcyclohexyl)ethan-1-one oxime was prepared and characterized as follows.

To a one neck, round bottom flask, was added 1-((1R,2R,4R)-2-hydroxy-4-methylcyclohexyl)ethan-1-one (4.0 g, 25.6 mmol) and ethanol (50 mL). While stirring at room temperature, pyridine (51.2 mmol) was added followed by hydroxylamine hydrochloride (28.2 mmol). The reaction mixture was stirred at room temperature until reaction was completed. The solvent was removed under reduced pressure. The residue was diluted with ethyl acetate and washed with water (1×) and brine (1×). The organic layer was then dried over magnesium sulfate, filtered, and concentrated under reduced pressure. The crude product was isolated by automated silica gel chromatography (Biotage Isolera, 6-50% ethyl acetate/hexanes) to afford 2.22 g (51%) of 1-((1R,2R,4R)-2-hydroxy-4-methylcyclohexyl)ethan-1-one oxime. ¹H NMR (400 MHz, CHCl₃-d) 6 ppm 0.95 (m, 6H) 1.22 (dd, J=12.82, 3.66 Hz, 1H) 1.49 (d, J=6.87 Hz, 1H) 1.68 (m, 2H) 1.85 (m, 4H) 2.03 (m, 2H) 3.71 (m, 1H). ¹³C NMR (101 MHz, CHCl₃-d) δ ppm 12.11, 22.18, 28.57, 31.39, 34.15, 42.55, 51.87, 70.46, 160.68

### Example 15: Synthesis of 1-((1R,2R,4R)-2-hydroxy-4-methylcyclohexyl)ethan-1-one O-methyl oxime

1-((1R,2R,4R)-2-hydroxy-4-methylcyclohexyl)ethan-1-one O-methyl oxime was prepared and characterized as follows.

The same procedure shown in Example 14 was followed to synthesize 1-((1R,2R,4R)-2-hydroxy-4-methylcyclohexyl)ethan-1-one O-methyl oxime using methoxyamine hydrochloride as a substrate. The yield for this reaction was 57%. ¹H NMR (400 MHz, CHCl₃-d) δ ppm 0.94 (m, 6H) 1.19 (dd, J=12.59, 3.43 Hz, 1H) 1.49 (d, J=6.41 Hz, 1H) 1.68 (m, 2H) 1.83 (m, 4H) 2.00 (m, 2H) 3.73 (m, 1H) 3.83 (s, 3H). ¹³C NMR (101 MHz, CHCl₃-d) δ ppm 12.78, 22.21, 28.56, 31.36, 34.24, 42.30, 51.88, 61.54, 70.30, 159.39

### Example 16: Synthesis of (1R,2S,5R)-2-((S)-1-hydroxyethyl)-5-methylcyclohexan-1-ol

(1R,2S,5R)-2-((S)-1-hydroxyethyl)-5-methylcyclohexan-1-ol was prepared and characterized as follows.

To a two neck, round bottom flask, equipped with a thermometer, was added 1-((1R,2R,4R)-2-hydroxy-4-methylcyclohexyl)ethan-1-one (20.0 g, 128 mmol) and anhydrous methanol (400 mL). The mixture was cooled to 0° C. then sodium borohydride (153.6 mmol) was added portion wise, keeping the internal temperature <5° C. The reaction mixture was continuously stirred at 0° C. until reaction was completed. The reaction was quenched with water. The solvent was removed under reduced pressure. The aqueous layer was extracted with ethyl acetate (3×). The combined organics were washed with brine (1×). The organic layer was then dried over magnesium sulfate, filtered, and concentrated under reduced pressure. The crude product was isolated by automated silica gel chromatography (Biotage Isolera, 20-100% ethyl acetate/hexanes) to afford 8.62 g (42.6%) of (1R,2S,5R)-2-((S)-1-hydroxyethyl)-5-methylcyclohexan-1-ol. GC/MS (EI): m/z (%) (Major isomer) 158 (M+) 143 (1) 140 (2) 125 (6) 122 (7) 111 (6) 107 (11) 96 (52) 81 (100) 67 (23) 55 (39) 54 (29) 45 (27) 43 (33) 41 (37). GC/MS (EI): m/z (%) (Minor isomer) 158 (M+) 143 (1) 140 (2) 125 (6) 122 (6) 111 (7) 107 (11) 96 (53) 81 (100) 67 (23) 55 (39) 54 (29) 45 (29) 43 (35) 41 (36). ¹H NMR (400 MHz, CHCl₃-d) δ ppm (Major+Minor isomers) 0.82-1.03 (m, 12H) 1.20 (m, 7H) 1.36-1.52 (m, 4H) 1.64 (m, 3H) 1.92 (m, 2H) 3.15 (d, J=5.95 Hz, 1H) 3.31 (br. s., 1H) 3.54 (td, J=10.19, 3.89 Hz, 1H) 3.70 (m, 2H) 3.94 (br. s., 3H). ¹³C NMR (101 MHz, CHCl₃-d) 6 ppm (Major isomer) 18.28, 22.11, 26.56, 31.14, 34.28, 44.36, 49.30, 70.85, 71.15. ¹³C NMR (101 MHz, CHCl₃-d) δ ppm (Minor isomer) 21.81, 27.07, 31.25, 34.00, 44.14, 50.18, 74.12, 76.27.

### Example 17: Synthesis of (7R)-4,7-dimethylhexahydro-4H-spiro[benzo[d][1,3]dioxine-2,1′-cyclohexane]

(7R)-4,7-dimethylhexahydro-4H-spiro[benzo[d][1,3]dioxine-2,1′-cyclohexane] was prepared and characterized as follows.

To a one neck, round bottom flask, equipped with a Dean-Stark trap and condenser, was added (1R,2S,5R)-2-((S)-1-hydroxyethyl)-5-methylcyclohexan-1-ol (Example 16) (2.0 g, 12.6 mmol) and toluene (30 mL). While stirring at room temperature, cyclohexanone (19.0 mmol) was added followed by pyridinium p-toluenesulfonate (1.2 mmol). The mixture was then refluxed until reaction was completed. The reaction mixture was then cooled to room temperature, diluted with ethyl acetate, and quenched with saturated NaHCO₃ (1×), water (1×) and brine (1×). The organic layer was then dried over magnesium sulfate, filtered, and concentrated under reduced pressure. The crude product was isolated by automated silica gel chromatography (Biotage Isolera, 2-20% ethyl acetate/hexanes) to afford 2.05 g (64%) of (7R)-4,7-dimethylhexahydro-4H-spiro[benzo[d][1,3]dioxine-2,1′-cyclohexane]. Odor description: fresh vanilla bean, leathery note, balsamic, phenolic sweetness. GC/MS (EI): m/z (%) (Major isomer) 238 (M⁺, 16) 209 (12) 196 (14) 195 (100) 182 (3) 140 (1) 123 (45) 112 (1) 99 (17) 81 (37) 69 (15) 67 (14) 55 (55) 41 (34). GC/MS (EI): m/z (%) (Minor isomer) 238 (M⁺, 17) 209 (11) 196 (15) 195 (100) 182 (3) 141 (2) 140 (1) 123 (61) 112 (3) 99 (16) 81 (50) 69 (23) 67 (20) 55 (89) 41 (51). ¹H NMR (400 MHz, CHCl₃-d) δ ppm (Major+Minor isomers) 0.82-1.05 (m, 7H) 1.11 (m, 2H) 1.20 (dd, J=6.87, 1.37 Hz, 1H) 1.33-1.67 (m, 11) 1.78 (m, 1H) 1.89 (br. s., 2H) 3.49 (m, 1H) 3.63 (m, 1H) 4.13 (m, 1H). ¹³C NMR (101 MHz, CHCl₃-d) δ ppm (Major isomer) 19.41, 22.36, 22.75, 22.78, 25.60, 26.00, 28.87, 31.45, 34.32, 39.15, 40.85, 47.80, 69.11, 71.84, 98.57. ¹³C NMR (101 MHz, CHCl₃-d) S ppm (Minor isomer) 18.07, 23.14, 23.34, 25.73, 25.82, 31.42, 34.65, 35.51, 37.80, 41.20, 43.94, 67.43, 68.73, 98.94

### Example 18: Synthesis of (7R)-4,7-dimethyl-2-(pentan-2-yl)hexahydro-4H-benzo[d][1,3]dioxine

(7R)-4,7-dimethyl-2-(pentan-2-yl)hexahydro-4H-benzo[d][1,3]dioxine was prepared and characterized as follows.

The same procedure shown in Example 17 was followed to synthesize (7R)-4,7-dimethyl-2-(pentan-2-yl)hexahydro-4H-benzo[d][1,3]dioxine using 2-methylpentanal as a substrate. The yield for this reaction was 49%. Odor description: mandarin-like, fatty, clean, cooling, citrus, linen fresh, mid range in strength. GC/MS (EI): m/z (%) (Major isomer) 240 (M⁺, 1) 239 (7) 198 (2) 169 (100) 141 (3) 123 (78) 107 (4) 96 (26) 81 (61) 67 (17) 55 (38) 43 (37) 41 (36). GC/MS (EI): m/z (%) (Minor isomer) 240 (M⁺, 1) 239 (6) 207 (3) 169 (99) 141 (8) 123 (100) 107 (5) 96 (12) 81 (64) 67 (19) 55 (43) 43 (44) 41 (44). ¹H NMR (400 MHz, CHCl₃-d) δ ppm (Major+Minor isomers) 0.80-0.95 (m, 19H) 1.05-1.25 (m, 11H) 1.43 (m, 6H) 1.61 (m, 5H) 1.89 (m, 2H) 3.17 (m, 1H) 3.33 (dtt, J=9.19, 6.11, 6.11, 3.26, 3.26 Hz, 1H) 3.49 (m, 1H) 4.09 (quin, J=6.53 Hz, 1H) 4.35 (m, 1H) 4.61 (m, 1H). ¹³C NMR (101 MHz, CHCl₃-d) δ ppm (Major isomer) 13.19, 14.28, 14.44, 20.21, 22.34, 25.60, 31.40, 34.22, 37.58, 40.26, 43.64, 47.27, 71.77, 73.88, 104.83. ¹³C NMR (101 MHz, CHCl₃-d) δ ppm (Minor isomer) 18.84, 14.23, 20.27, 26.24, 33.67, 33.85, 33.89, 33.94, 34.46, 37.48, 40.76, 80.20, 97.06, 97.31

### Example 19: Synthesis of (7R)-2-(tert-butyl)-4,7-dimethylhexahydro-4H-benzo[d][1,3]dioxine

(7R)-2-(tert-butyl)-4,7-dimethylhexahydro-4H-benzo[d][1,3]dioxine was prepared and characterized as follows.

The same procedure shown in Example 17 was followed to synthesize (7R)-2-(tert-butyl)-4,7-dimethylhexahydro-4H-benzo[d][1,3]dioxine using trimethyl acetaldehyde as a substrate. The yield for this reaction was 69%. Odor description: strong, supple leather, leathery note, clean, oily. GC/MS (EI): m/z (%) (Major isomer) 226 (M⁺, 0.5) 225 (4) 169 (78) 141 (6) 123 (100) 107 (4) 96 (11) 81 (63) 67 (19) 55 (34) 41 (44). GC/MS (EI): m/z (%) (Minor isomer) 226 (M⁺, 0.8) 225 (5) 169 (98) 141 (2) 123 (100) 107 (5) 96 (31) 81 (84) 67 (24) 55 (44) 41 (60). ¹H NMR (400 MHz, CHCl₃-d) δ ppm (Major+Minor isomers) 0.89 (m, 20H) 1.04 (m, 2H) 1.17 (m, 5H) 1.40 (m, 2H) 1.57 (d, J=0.92 Hz, 1H) 1.63 (m, 3H) 1.88 (br. s., 1H) 3.15 (td, J=10.42, 3.89 Hz, 1H) 3.30 (m, 1H) 3.47 (m, 1H) 4.08 (quin, J=6.64 Hz, 1H) 4.15 (d, J=0.92 Hz, 1H) 4.40 (s, 1H). ¹³C NMR (101 MHz, CHCl₃-δ) ppm (Major isomer) 13.19, 22.36, 24.85, 26.23, 31.37, 34.49, 40.80, 43.56, 47.19, 71.68, 73.83, 107.03. ¹³C NMR (101 MHz, CHCl₃-d) δ ppm (Minor isomer) 18.86, 24.94, 25.59, 31.40, 34.26, 34.76, 40.33, 76.99, 80.10, 99.59

### Example 20: Synthesis of (7R)-2-isopropyl-4,7-dimethylhexahydro-4H-benzo[d][1,3]dioxine

(7R)-2-isopropyl-4,7-dimethylhexahydro-4H-benzo[d][1,3]dioxine was prepared and characterized as follows.

The same procedure shown in Example 17 was followed to synthesize (7R)-2-isopropyl-4,7-dimethylhexahydro-4H-benzo[d][1,3]dioxine using isobutyraldehyde as a substrate. The yield for this reaction was 33%. Odor description: cool, rhubarb, grapefruit, yellow fruit. GC/MS (EI): m/z (%) (Major isomer) 212 (M⁺, 0.9) 211 (6) 169 (73) 141 (6) 123 (100) 107 (5) 96 (13) 81 (70) 67 (21) 55 (40) 43 (41) 41 (42).

GC/MS (EI): m/z (%) (Minor isomer) 212 (M⁺, 1) 211 (9) 169 (96) 141 (2) 123 (100) 107 (7) 96 (43) 81 (99) 67 (28) 55 (54) 43 (57) 41 (60). H NMR (400 MHz, CHCl₃-d) 6 ppm (Major+Minor isomers) 0.85-1.09 (m, 17H) 1.15-1.21 (m, 4H) 1.41 (m, 2H) 1.69 (m, 4H) 1.89 (m, 1H) 3.17 (m, 1H) 3.33 (m, 1H) 3.48 (td, J=10.76, 4.12 Hz, 1H) 4.08 (quin., J=1.00 Hz, 1H) 4.26 (d, J=5.50 Hz, 1H) 4.51 (d, J=5.50 Hz, 1H). ¹³C NMR (101 MHz, CHCl₃-δ) ppm (Major isomer) 13.20, 17.20, 17.41, 22.34, 26.23, 31.37, 32.74, 34.45, 40.74, 43.60, 71.73, 73.82, 105.53. ¹³C NMR (101 MHz, CHCl₃-d) δ ppm (Minor isomer) 17.46, 17.50, 18.82, 25.58, 31.39, 32.78, 34.21, 40.23, 47.23, 80.14, 97.96

### Example 21: Synthesis of (7R)-2-ethyl-4,7-dimethylhexahydro-4H-benzo[d][1,3]dioxine

(7R)-2-ethyl-4,7-dimethylhexahydro-4H-benzo[d][1,3]dioxine was prepared and characterized as follows.

The same procedure shown in Example 17 was followed to synthesize (7R)-2-ethyl-4,7-dimethylhexahydro-4H-benzo[d][1,3]dioxine using propionaldehyde as a substrate. The yield for this reaction was 65%. Odor description: balsamic, oily, sweet, strong, little animalic, touch of costus. GC/MS (EI): m/z (%) (Major isomer) 198 (M⁺, 1) 197 (7) 169 (71) 141 (8) 123 (100) 107 (8) 96 (18) 81 (84) 67 (23) 55 (41) 41 (39). GC/MS (EI): m/z (%) (Minor isomer) 198 (M⁺, 1) 197 (9) 169 (68) 141 (3) 123 (73) 107 (7) 96 (45) 81 (100) 67 (25) 55 (45) 41 (44). H NMR (400 MHz, CHCl₃-d) δ ppm (Major+Minor isomers) 0.82-1.09 (m, 9H) 1.15-1.21 (m, 3H) 1.39-1.63 (m, 5H) 1.75 (m, 1H) 1.88 (m, 1H) 3.19 (ddd, J=11.22, 9.62, 3.89 Hz, 1H) 3.35 (dq, J=9.39, 6.18 Hz, 1H) 3.50 (td, J=10.76, 4.12 Hz, 1H) 4.08 (quin, J=6.64 Hz, 1H) 4.51 (t, J=5.27 Hz, 1H) 4.75 (t, J=5.04 Hz, 1H). ¹³C NMR (101 MHz, CHCl₃-δ) ppm (Major isomer) 8.56, 13.19, 22.31, 26.22, 28.31, 31.36, 34.42, 40.71, 43.53, 71.82, 73.80, 102.82. ¹³C NMR (101 MHz, CHCl₃-d) δ ppm (Minor isomer) 8.73, 18.80, 25.56, 28.21, 34.17, 40.19, 47.15, 80.13, 95.33

### Example 22: Synthesis of 1-((1R,2R,4R)-4-methyl-2-((tetrahydro-2H-pyran-2-yl)oxy)cyclohexyl)ethan-1-one

1-((1R,2R,4R)-4-methyl-2-((tetrahydro-2H-pyran-2-yl)oxy)cyclohexyl)ethan-1-one was prepared and characterized as follows.

To a one neck, round bottom flask, equipped with a condenser, was added 1-((1R,2R,4R)-2-hydroxy-4-methylcyclohexyl)ethan-1-one (10.0 g, 64.0 mmol) and dichloromethane (125 mL). While stirring at room temperature, 3,4-dihydro-2H-pyran (128 mmol) was added followed by pyridinium p-toluenesulfonate (6.4 mmol). The mixture was stirred at room temperature for two hours then heated at 55° C. until reaction was completed. The reaction mixture was then cooled to room temperature, diluted with ethyl acetate, and quenched with water (1×). The organic layer was then dried over magnesium sulfate, filtered, and concentrated under reduced pressure. The crude product was isolated by automated silica gel chromatography (Biotage Isolera, 6-50% ethyl acetate/hexanes) to afford 10.97 g (71%) of 1-((1R,2R,4R)-4-methyl-2-((tetrahydro-2H-pyran-2-yl)oxy)cyclohexyl)ethan-1-one. GC/MS (EI): m/z (%) (Major isomer) 240 (M*) 156 (4) 140 (19) 139 (24) 122 (5) 101 (12) 95 (14) 85 (65) 67 (13) 55 (15) 43 (100) 41 (23). GC/MS (EI): m/z (%) (Minor isomer) 240 (M⁺) 156 (4) 155 (4) 140 (17) 139 (25) 122 (4) 101 (9) 95 (14) 85 (71) 67 (14) 55 (16) 43 (100) 41 (24). H NMR (400 MHz, CHCl₃-d) δ ppm (Major+Minor isomers) 0.76-1.07 (m, 9H) 1.25-1.80 (m, 22) 2.12 (m, 4H) 2.23 (d, J=2.29 Hz, 3H) 2.50 (m, 2H) 3.44 (m, 2H) 3.69 (m, 2H) 3.85 (m 2H) 4.54 (m, 1H) 4.76 (br. s., 1H). ¹³C NMR (101 MHz, CHCl₃-d) δ ppm (Major isomer+Minor isomers) 19.33, 20.32, 22.12, 22.23, 25.40, 25.43, 27.85, 30.85, 30.91, 31.00, 31.17, 31.26, 33.51, 33.63, 38.83, 42.36, 56.50, 56.87, 61.95, 63.34, 74.09, 79.92, 93.74, 101.55, 212.47, 212.57

### Example 23: Flavor Evaluations

The present Example provides the evaluation of various compounds of the presently disclosed subject matter. Table 1 provides a summary of the compound, number of panelists, concentration of compound and flavor comments using a “swish and spit” protocol described in Example 24.

TABLE 1

| | # OF | CONCEN- | |
|---|---|---|---|
| | PANEL- | TRATION | |
| COMPOUND | ISTS | (PPM) | COMMENTS |
| (1R,2R,5R)-2-acetyl-5- | 4 | 10 | Slight sensation, |
| methylcyclohexyl | | | gluey, bacteria- |
| propionate | | | like off note |
| (1R,2R,5R)-2-acetyl-5- | 3 | 50 | Slightly fruity, |
| methylcyclohexyl | | | bitter, metallic, |
| butyrate | | | caproate notes |
| (1R,2R,5R)-2-acetyl-5- | 3 | 50 | Bitter, fruity |
| methylcyclohexyl | | | note, slight |
| isobutyrate | | | mushroom, old |
| | | | cucumber |
| (1R,2R,5R)-2-acetyl-5- | 3 | 50 | Sharp bitterness |
| methylcyclohexyl | | | |
| pentanoate | | | |
| (1R,2R,5R)-2-acetyl-5- | 3 | 50 | Bitter, slight |
| methylcyclohexyl | | | cloying |
| cyclopropanecarboxylate | | | sensation |
| (1R,2R,5R)-2-acetyl-5- | 5 | 10 | Very bitter, glue |
| methylcyclohexyl | | | like taste, dairy |
| cyclohexanecarboxylate | | | |
| (1R,2R,5R)-2-acetyl-5- | 5 | 10 | Bitter, fruity, |
| methylcyclohexyl 2- | | | cucumber, slight |
| methylbutanoate | | | apple taste that |
| | | | lingers, melon |
| | | | rind, fatty |
| (1R,2R,5R)-2-acetyl-5- | 5 | 10 | Slight cooling, |
| methylcyclohexyl | | | salivation, |
| methacrylate | | | mushroom, |
| | | | dirty, coconut, |
| | | | metallic |
| (1R,2R,5R)-2-acetyl-5- | 5 | 10 | Fruity, |
| methylcyclohexyl | | | extremely bitter, |
| cyclopentanecarboxylate | | | sour grapes, |
| | | | fermented |
| (1R,2R,5R)-5-methyl-2- | 5 | 100 | Bitter, weak, |
| (2-methyl-1,3-dioxolan-2- | | | envelope glue |
| yl)cyclohexan-1-ol | | | |
| (1R,2R,5R)-5-methyl-2- | 7 | 30 | Bitter, |
| (2,5,5-trimethyl-1,3- | | | astringent |
| dioxan-2-yl)cyclohexan- | | | |
| 1-ol | | | |
| (1R,2R,5R)-2-((2R)-4- | 7 | 30 | Bitter, slight |
| ethyl-2-methyl-1,3- | | | cooling, fruity, |
| dioxolan-2-yl)-5- | | | creamy, slight |
| methylcyclohexan-1-ol | | | herbal, woody, |
| | | | pineapple |
| (1R,2S,5R)-2-((S)-1- | 7 | 30 | Bitter, weak, |
| hydroxyethyl)-5- | | | fruity, cheesy |
| methylcyclohexan-1-ol | | | |

### Example 24: Perceived Bitter Masking in Caffeine Solution

The present Example provides the perceived bitter masking of (1R,2R,5R)-2-acetyl-5-methylcyclohexyl methacrylate (Example 8) of the presently disclosed subject matter.

A caffeine solution was prepared for use as a bitterness reference sample by adding 0.20 g of neat caffeine powder to 1 L of water resulting in solution with 200 ppm of caffeine (0.02%). Another solution was prepared by adding 0.02 g of (1R,2R,5R)-2-acetyl-5-methylcyclohexyl methacrylate (which has been diluted to 1% in ethanol) in 100 g of the caffeine solution. The caffeine composition with 2 ppm (0.0002%) of (1R,2R,5R)-2-acetyl-5-methylcyclohexyl methacrylate was evaluated and compared to the caffeine composition without additives for its perceived bitter masking in the mouth by 3 expert evaluators following a “swish and spit” protocol, in which the sample is placed into a small tasting cup, taken into the mouth, and swished around, then the sample is spit back into the original tasting cup and disposed of.

All evaluators perceived the composition with (1R,2R,5R)-2-acetyl-5-methylcyclohexyl methacrylate as having less bitterness, and a slight metallic finish than the composition without.

### Example 25: Fragrance Composition

The present Example provides a fragrance formula for a Citrus Marine Men's Cologne. Table 2 provides a summary of the category of components of the fragrance formula.

TABLE 2

| CATEGORY | PARTS PER THOUSAND |
|---|---|
| ALDEHYDIC | 3.0 |
| AMBER | 1.5 |
| EXAMPLE 20 - CITRUS | 100.0 |
| CITRUS | 178 |
| FLORAL | 262.3 |
| FLORAL/ANIMALIC | 1.0 |
| FRUITY | 3.5 |
| GREEN | 11.8 |
| HERBACEOUS | 1.0 |
| MARINE | 2.0 |
| MOSSY | 1.0 |
| MUSK | 7.0 |
| PINEY | 1.2 |
| SOLVENT | 64.2 |
| SPICY | 4.0 |
| WOODY | 30.0 |
| WOODY/AMBER | 328.5 |
| | 1000 |

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method, and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1. A compound represented by Formula I: 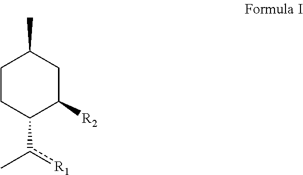
wherein R₁ is one of O, —OH, —NOH—NOR, or NOC(O)R;
wherein R₂ is one of —OC(O)R, —OH, or —OTHP; and
wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

2. The compound of claim 1, wherein R₁ is one of —OH, —NOH—NOR, or NOC(O)R; and wherein R₂ is —OH.

3. A compound represented by Formula Ia: 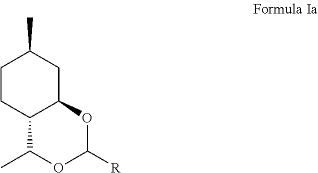
wherein R is selected from the group consisting of C₁-C₈ alkyl, C₁-C₈ branched alkyl, C₁-C₈ alkenyl, C₁-C₈ branched alkenyl, C₁-C₈ cycloalkyl, aryl and substituted aryl.

4. A compound represented by Formula Ib: 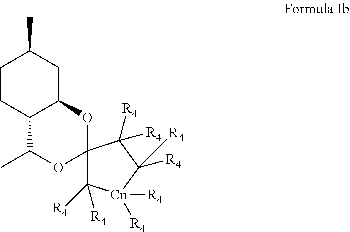
wherein n is 1, 2, 3, or 4;
wherein each R₄ is independently H or C₁-C₈ alkyl.

5. (canceled)

6. (canceled)

7. A fragrance composition comprising at least one compound of claim 1.

8. The fragrance composition of claim 7, wherein the concentration of the at least one compound is from about 0.001% to about 20% by weight of the fragrance composition.

9. The fragrance composition of claim 7, further comprising one or more compounds selected from the group consisting of one or more aldehydic compound(s), one or more animalic compound(s), one or more balsamic compound(s), one or more citrus compound(s), one or more floral compound(s), one or more fruity compound(s), one or more gourmand compound(s), one or more green compound(s) one or more herbaceous compound(s) one or more marine compound(s), one or more mossy compound(s), one or more musk compound(s), one or more piney compound(s), one or more powdery compound(s), one or more spicy compound(s) and/or one or more woody compound(s), and combinations thereof.

10. A flavor composition comprising at least one compound of claim 1.

11. The flavor composition of claim 10, wherein the concentration of the at least one compound is from about 0.0001% to about 20% by weight of the flavor composition.

12. The flavor composition of claim 10, further comprising a flavor carrier.
